(19) United States
(12) Patent Application Publication
(10) Pub. No.: US 2022/0088045 A1
(43) Pub. Date: Mar. 24, 2022

(54) Treatment compositions

(71) Applicant: Mo S. Kharazmi, Trabuco Canyon, CA (US)

(72) Inventor: Mo S. Kharazmi, Trabuco Canyon, CA (US)

(21) Appl. No.: 17/483,654

(22) Filed: Sep. 23, 2021

Related U.S. Application Data
(60) Provisional application No. 63/082,428, filed on Sep. 23, 2020.

(51) Int. Cl. A61K 31/706; A61K 31/09; A61K 31/12

(52) U.S. Cl. CPC A61K 31/706; A61K 31/12; A61K 31/09

(57) ABSTRACT

In embodiments, the invention includes compositions and methods of administering the compositions. The compositions include one or more active components, which may be encapsulated and dried. The encapsulated components may be introduced into water to form a beverage.

### RELATED PATENT APPLICATION

This application claims the benefit of U.S. provisional patent application No. 63/082,428 entitled Treatment compositions, filed on Sep. 23, 2020, by Mo S. Kharazmi. The entire content of this provisional patent application is incorporated by reference herein for all purposes.

Title: Treatment compositions

Inventor: Mo S. Kharazmi

### BACKGROUND

### Field

This invention is in the field of treatment of molecular-scale injury due to aging, degeneration, or disease. It includes compositions and methods of administering the compositions.

There is a need for improved methods and compositions to treat health-related problems of aging using methods of demonstrable efficacy to remodel and repair impaired or aged tissue and cellular components.

Nicotinamide adenine dinucleotide (NAD) is an important enzyme cofactor involved in numerous redox reactions in the body. Its level in tissues declines with age and this decline appears correlated with age-related changes such as stiffening of blood vessels, oxidative stress, cellular senescence, and changes in gene expression. Sirtuins are a family of NAD-dependent enzymes that regulate many regulatory proteins for metabolism, DNA repair, stress response, chromatin remodeling, and other cellular processes. This suggests that the effects of NAD level decrease may cascade to produce many symptoms of aging. There is thus a need to increase NAD levels to help reduce age-related defects.

NAD can be synthesized de novo by the conversion of the amino acid tryptophan through multiple enzymatic steps to nicotinic acid mononucleotide. There is also a salvage pathway for NAD production limited by the activity of the enzyme nicotinamide phosphoribosyltransferase, which converts nicotinamide to nicotinamide mononucleotide (NMN). The extracellular uptake of NAD is low, so supplementation with NAD is ineffective, but a specific transporter enables uptake of NMN, suggesting that supplementation with NMN may bypass the rate limiting synthesis steps to raise NAD levels.

Low levels of NAD are not the sole cause of aging, and its replacement is not a cure. There is thus a need for a combination supplementation that addresses more aged-related symptoms.

### SUMMARY

I have found that a combination of active components can reduce many of the effects of aging. My invention includes a combination of active components that may be encapsulated. The encapsulation helps prevent degradation of the active components during storage and may also help prevent degradation during digestion and transport to the tissues.

The active components include one or more of NMN, pterostilbene, and xanthohumol in effective amounts. The active components may be stored as a dry combination and mixed with water shortly before use. This combination of active components and water helps hydration and provides a simple method of consuming the active components.

In embodiments, the invention includes a composition comprising NMN, pterostilbene, and xanthohumol. The composition may include about 25-500 mg NMN, about 10-100 mg pterostilbene, and about 4-100 mg xanthohumol. In some embodiments, the composition may be suspended or dissolved in 200-1000 mL water.

In embodiments, one or more of the NMN, the pterostilbene, and the xanthohumol may be encapsulated. One or more of the NMN, the pterostilbene, and the xanthohumol may be dried, either before or after encapsulation. The dried and/or encapsulated composition of the NMN, the pterostilbene, and the xanthohumol may be suspended or dissolved in water.

In other embodiments, the invention includes a composition comprising an aqueous suspension of an encapsulated active component, where the active component includes one or more of NMN, pterostilbene, and xanthohumol. The encapsulated active component may have a capsule median diameter in the range of about 20 nm to about 500 nm.

The active component may include two or more of NMN, pterostilbene, and xanthohumol. In other embodiments, the active component may include each of NMN, pterostilbene, and xanthohumol. At least two of the NMN, the pterostilbene, and xanthohumol may be separately encapsulated.

In any of these embodiments, the encapsulated active component may comprise a capsule including one or more of a liposome or a polymer capsule. The polymer capsule may include one or more of an alginate, a polysaccharide, a protein, a synthetic polymer, or a derivative of any of these as well as other materials.

In embodiments, the capsule may also include a targeting material.

The invention also includes a method of administering a treatment composition. This method includes the steps of providing a first composition having a dry encapsulated active component. The active component may include one or more of NMN, pterostilbene, and xanthohumol. The method also includes the step of suspending a predetermined amount of the first composition in a predetermined volume of water to form a beverage; and the step of drinking the beverage to administer the composition.

In embodiments, the predetermined volume may be about 200 to about 1000 mL. The first composition may include an unencapsulated active component. The unencapsulated active component includes a flavoring or an electrolyte. An electrolyte may be present as a salt.

### DETAILED DESCRIPTION

The invention includes a treatment composition and a method of administering the treatment composition.

The treatment composition of the invention includes one or more active components that may be encapsulated. An effective amount of the treatment components may be combined with water to form a beverage. The beverage may also include flavorings such as citric acid and electrolytes such as carbonate and calcium.

Active components may include nicotinamide adenine dinucleotide (NAD), nicotinamide mononucleotide (NMN), pterostilbene, xanthohumol, or a combination of these.

In embodiments, the active components include an effective amount of NMN. This amount may be in the range of 25-500 mg. In some embodiments, the active components include an effective amount of pterostilbene. This amount may be in the range of 10-100 mg. In embodiments, the active components include an effective amount of xantohumol. This amount may be in the range of 4-100 mg.

In some embodiments, the invention includes a combination of 25-500 mg NMN, 10-100 mg pterostilbene, and 4-100 mg xantohumol. The combination may be suspended in 200-1000 mL of water.

Encapsulation

In some embodiments, components of the treatment compositions may be encapsulated. Encapsulation can protect active agents from surrounding environments. Encapsulation may also serve to control the release of active materials over a desired time (e.g. when exposed to a particular environment in a bottle containing water, in the digestive tract or in blood or tissue) or at a desired rate.

Encapsulation may also serve to aid in delivering particular components to targeted cells or to targeted compartments in cells where the components can directly act on cellular targets such as nucleic acids or oxidative enzymes.

Encapsulation may be performed by any of a number of methods known in the art. Conventional encapsulation processes, such as encapsulation by liposomes may be used, as well as other methods, such as those reviewed by Yadav et al. in Peptides 32 173-187 (2011). This review is hereby incorporated by reference for its disclosure of methods of encapsulation.

Individual treatment components may be mixed before encapsulation or may be encapsulated separately. In some embodiments, treatment compositions include lipophilic materials. Encapsulation of lipophilic materials may require different conditions from encapsulation of aqueous materials. In such embodiments, the treatment compositions may include a combination of at least two sets of encapsulated materials.

Encapsulation processes may differ in the size of the capsules produced. Capsules are usually not monodisperse but have a range of sizes. An encapsulated product where the capsules are prepared in a common process may be characterized by a size distribution having a median capsule diameter. In embodiments, the capsule median diameter may be in the range of about 20 nm to about 500 nm or larger. An advantage of the range of capsule sizes is that the contents of the capsules may be released over an extended period of time because the different size capsules release their content at different times or rates.

In this document, a component is nanoencapsulated if its capsule median diameter is smaller than about 100 nm. Components in capsules with capsule median diameter greater than about 100 nm are microencapsulated. A composition may include a combination or mixture of capsule sizes, including different components that are microencapsulated and nanoencapsulated. A composition may also include a combination of different size capsules containing the same component.

Capsules may release their contents when broken or ruptured. In some situations, components may escape through the walls of intact capsules. This is particularly applicable in thin wall capsules such as liposomes. Capsules may have walls composed of polymers or lipids that melt at body temperatures or have a glass transition temperature below body temperature, so that such capsules may release their contents after ingestion. Capsules may rupture due to mechanical forces or temperature or osmotic pressure following ingestion of the treatment composition. Capsules may also be sensitive to chemical conditions in tissue such as pH or the presence of reactive oxygen species (ROS). In other embodiments, the treatment compositions may be subjected to externally applied heat, pressure, or vibration to aid in capsule content release.

In some embodiments, capsules may include targeting materials. Targeting materials are moieties accessible on capsule surfaces that engage cellular receptors to promote uptake of the capsules or their contents by the cells or by cellular organelles.

Examples of targeting materials include cell-penetrating peptides, which also referred to as protein-transduction domains. Cell-penetrating peptides are a diverse set of membrane active peptides of fewer than about 30 residues, commonly with a net positive charge. These interact with eucaryotic membrane components to generate pores through which associated cargos may enter the cells. Such cell-penetrating peptides are known to facilitate the delivery of various biomolecules across cellular membranes of eukaryotic cells with limited toxicity. The molecular weight of the bioactive cargo, which may be linked covalently or noncovalently, may be several times greater than the molecular weight of the cell-penetrating peptides and may include liposomes. Certain cell-penetrating peptides are described in a review by FG Avci et al. in Biomolecules 8: 77 (2018) doi: 10.3390/biom8030077. This review and its cited references numbered 67-71 and 82 are hereby incorporated by reference for their disclosure of cell-penetrating peptides and their use.

In other embodiments, targeting materials may include nutrients that are selectively taken up by cells. For example, a targeting material may include phenylalanine, an essential amino acid.

In other embodiments, targeting materials may include specific tags such as the CKGGRAKDC peptide that specifically binds to white fat vasculature. Encapsulated components bearing such specific tags on their surfaces may bind to the cellular targets and thus be delivered to particular cell types. Other exemplary specific tags include the KNESSTNATNTKQWRDETKGFRDEAKRFKNTAG peptide derived from T7 phage p17 tail fiber. See S C Wong, et al. in Molecular Pharmaceutics 3, 4 386-97 (2006), which is hereby incorporated by reference for its disclosure of a tag that selectively binds to hepatocytes and its use for targeting liposome payloads to such cells.

In still other embodiments, targeting materials may include tags specific for particular organelles within cells. For example, mitochondrial import stimulating factors include Tom (transporter outer membrane) binding sequences: these include 10-70 amino acid long peptide that directs a newly synthesized protein to the mitochondria. The sequences consist of an alternating pattern of hydrophobic and positively charged amino acids. Nanoencapsulated components bearing such specific tags on their surfaces may bind to the subcellular targets and thus be delivered to particular portions of cells. This may be of particular benefit when the active composition includes NAD+ or NMN. Similar import factors are known for nucleus, endoplasmic reticulum, and other cellular structures. Those specific for delivery to the nucleus may be of particular interest for treatment components that are intended to alter DNA methylation.

A targeting material may be bound covalently or noncovalently to the capsule. In the case of a liposome, the targeting material may be modified by inclusion of a hydrophobic tail that dissolves into the liposome membrane so that at least some of the targeting material extends outwardly from the liposome surface.

In some embodiments, a suitable method of encapsulation includes emulsification polymerization using aqueous phase methacrylate monomer and a photoinitator such as benzoin ethyl ether emulsified with a treatment component with polyethylene oxide as a stabilizer and exposure to UV light after emulsification to produce a poly(methacrylate) encapsulated treatment component. The capsules may range from about 50 to about 3000 nm in diameter. While the capsules may be close to monodisperse (depending on details of the method of emulsification), in some embodiments, the size of capsules may be deliberately widely distributed to control the rate of release of active materials. Widely distributed populations of capsules may be prepared by altering the conditions of emulsification during encapsulation or by mixing two or more batches of capsules with different size.

In other embodiments, methods of encapsulation may also include multilayer prilling techniques. As used herein, prilling refers to a method of producing reasonably uniform spherical particles from solutions or suspensions and surrounding them with dissolved polymers. Prilling essentially consists of two operations typically performed in a sheathed flow stream. Liquid droplets containing the material to be encapsulated by sheathing them with dissolved polymers (or in some cases, molten polymers or monomers with polymerization agents). The sheath forms a shell surrounding the droplets as the sheath material desolvates or solidifies or polymerizes. The size of the capsules may be controlled by adjusting flow rates and droplet breakup rate. One commercial device available for such encapsulation is the Encapsulator B-390, manufactured by BUCHI Labortechnik AG of Flawil Switzerland.

In other embodiments, encapsulated treatment component may be prepared using the apparatus and method described in US patent publication 2008/0182019. This publication is hereby incorporated by reference for its disclosure of methods of encapsulation of aqueous phase materials. The described method produces hollow capsules of polymer surrounding aqueous materials in nested sheath flows. The size of the capsules and the thickness of the capsule walls are determined by the concentrations of polymers in the solvent and by the relative flow rates of the core and sheath streams. The polymer may be dissolved in a compatible solvent. In embodiments, the polymer may be a polylactide, a polycaprolactone, a poly glycolic acid, a poly methacrylate polymer, or other soluble polymers. The first three of these are biodegradable and will break down once ingested in the body, releasing the treatment component contents of the capsule. In particular, polycaprolactone may be degraded by hydrolysis of its ester linkages under physiological conditions. By controlling the size and wall thickness of the capsules (or mixtures of different size or material capsules), the treatment component may be released at a controlled rate, extending the effective time of treatment by making the treatment component available over an extended interval.

In other embodiments, a suitable method of encapsulation mixes the treatment component with a capsule forming material at a low concentration, divides the mixed treatment component-polymer combination into discrete droplets, cures or develops a capsule wall at the boundary of the droplets by exposure to a curing bath, and then extracts or removes residual capsule forming material from the capsules. A number of wash steps (relying on sedimentation, filtration, or mild centrifugation) may be used to achieve desired properties.

Division of the treatment component with a capsule forming material may be achieved by any of the methods discussed or referenced above and include discrete droplet formation or addition by dispensing through air or nonreactive solvent, aerosolized or ultrasonic stream breakup, or emulsification as a dispersed phase into a relatively immiscible suspending phase. In the latter case, exogenous surfactants may be added. Alternatively, components of the treatment component may serve as surfactants to stabilize the dispersed phase long enough for capsule formation. Emulsification may be accomplished by controlled mixing of the dispersed and suspending phases, such as pumping the materials between two syringes connected by a small diameter needle.

In one embodiment of this type, the capsule forming material may be an alginate solution, such as about 0.6 percent sodium alginate solution mixed in the treatment component and the curing bath may include about 1.5 percent calcium chloride solution, which causes the alginate to gel. The gelled capsules may be decanted and further treated in a polylysine solution (0.02 percent; 35 kD) for three to five minutes. The polylysine alginate microcapsules may then be washed with 1 percent calcium chloride solution, resuspended in an aqueous polyethyleneimine solution (0.2 percent; 40-60 kD) for 3 minutes, and then washed again with calcium chloride followed by isotonic saline. The encapsulated treatment component may then be suspended in isotonic sodium citrate solution, pH 7.4, for about 5 minutes in order to liquefy the alginate gel inside the capsule.

In one embodiment of this type, the capsule forming material may be a 1.5% solution of soluble sodium cellulose sulfate mixed in the treatment component and the curing bath may include a cationic polymer such as 0.85% polydiallyldimethylammonium (pDADMAC) solution in 0.9% NaCl. The reaction time in the curing bath may be about two minutes. The capsules may then be washed in normal saline to remove residual pDADMAC.

In other embodiments, components may be prepared in polymer capsules. The materials used for encapsulation with polymers may be selected from conventional hydrophilic or hydrophobic substances or mixtures thereof. Solids, in particular natural polymers, for example, starch and other polysaccharides, or zein or other proteins, are preferred because of their biocompatibility. However, synthetic polymers can also be used. Examples of shell materials are fats and/or waxes, preferably those having a solidification temperature of approximately 30-80 Celsius and include mixtures of cetyl palmitate and cetyl alcohol. Other useful shell compounds include: polysaccharides and their derivatives of natural or partially synthetic origin, (e.g. cellulose derivatives); polymers of γ- and/or β-hydroxycarboxylic acids, in particular polymers of glycolic acid (polyglycollides), lactic acid (polylactides), γ-hydroxybutyric acid (polyhydroxybutyrate), γ-hydroxyvaleric acid (poly (3-hydroxyvalerate) and/or their copolymers, or mixtures of such polymers and/or copolymers.

Hydroxy methylcellulose or other polysaccharides or zein, albumin, gelatin or other proteins core-shell capsules may be prepared by a coacervation technique. The shell material is dissolved in an aqueous solvent in a colloidal or a true solution. An organic-phase core material to be packaged (such as the relatively hydrophobic pterostilbene) may be dissolved in a suitable organic solvent such as coconut oil or food grade mineral oil. This is dispersed with the shell material in the form of solids or microdroplets. The dispersion may be divided into microdroplets and then heated with hot air or dried under reduced pressure. The aqueous solvent evaporates and the shell material reprecipitates in as a solid or gel, forming a shell around the core material. Again, the capsule size range depends on the precise conditions used in a manner familiar to those skilled in the art.

In some embodiments, the capsules include liposomes. Liposomes contain one or more lipophilic surfactants such as dipalmitoylphosphatidylcholine or phosphatidylinositol. Cholesterol may be added as a further liposome component to improve stability. Dipalmitoylphosphatidylcholine is a phospholipid consisting of two palmitic acids attached of a phosphatidylcholine head-group. Phosphatidylinositol is a phosphatidylglyceride including an inositol group. These materials are merely illustrative of a class of lipophilic surfactants such as occur in cell membranes. One or more of these lipophilic surfactant materials (or a mixture of the materials with cholesterol) may be mixed with pterostilbene in organic solvent with the pterostilbene forming between 0.5 and 10% of the weight of the mixture. After drying the solvent, the residue may be mixed with phosphate buffered saline (120 mM, pH 7) and extruded through 400 nm pore sized polycarbonate filters to form liposomes. Liposomes may also be used to encapsulate aqueous phase materials by replacing the organic solvent with an aqueous buffer.

The size of the liposomes produced depends on mechanical parameters of the process, such as the pore size of the filters and the flow rate through the pores. Size distribution may be adjusted by varying these parameters.

Preferred size range for liposomes or other capsules is in the range of about 20 nm to about 500 nm. In some embodiments, the range may about 50 nm to about 250 nm. This narrower size range beneficially delivers a more consistent dosing over time. Smaller particles divide the total dose of packaged materials into more liposomes, thereby decreasing the average distance between liposomes and reducing diffusion time.

In some embodiments, the capsules are formed to subcellular dimensions. For example, capsules may be made from 20 nm to about 500 nm in average size to facilitate uptake of the capsules or their contents by individual cells. This is of particular value when the capsule include targeting materials.

A benefit of the encapsulation of treatment composition components is that the encapsulated component may gradually extract from the capsules, making the component available in solution over an extended time. A still further benefit that the encapsulated component acts as a reservoir to “buffer” the component concentration in the in contact with tissue to a relatively constant sustained value.

In other embodiments, microencapsulated aqueous components such as nicotinamide mononucleotide may be prepared by similar methods to that described above for polymer capsules containing lipophilic materials except that the phases are reversed so that the to be encapsulated materials are dispersed in the aqueous phase. The size may be adjusted by varying the physical parameters such as proportion of lipophilic to hydrophilic phases, mixing stringency for coacervation techniques, or emulsification parameters for emulsification polymerization or interface polymerization.

The concentrations and conditions described for these embodiments are merely exemplary; those skilled in the art of microencapsulation will recognize that concentrations and conditions may be varied. Some routine optimization may be necessary for given lots, polymer size, and concentrations of materials.

Encapsulated treatment components may be washed by dialysis, by centrifugal filtration, by tangential flow filtration, by centrifugation and decanting, or by other techniques known in the art, to produce washed encapsulated treatment component. Washing helps remove unreacted monomers or initiator as well as materials not incorporated into capsules. Alternatively, and depending on the materials used in the encapsulation process, an encapsulated treatment component may be used without further processing. After washing, encapsulated treatment component may be resuspended in a carrier such as buffer, sterile saline, water, or in a suspension containing other materials. In other embodiments, the encapsulated treatment materials, or a combination of multiple encapsulated treatment materials, may be stored in a dry state. The dry materials may be combined with water shortly before use so that the treatment composition in water has a relatively consistent composition irrespective of variations in storage time.

In some embodiments, treatment component may be washed by mixing the encapsulated treatment component with two to four times the treatment component volume of a wash solution, which may be a normal saline solution or similar biologically compatible liquid. The treatment component and wash solution may be mixed in a centrifugal concentrator, such as a Vivaspin 20 Centrifugal Concentrator, with a 0.2 μm polyethersulfone membrane produced by Sartorius AG of Goettingen, Germany. The concentrator containing the mixture may then be centrifuged and the concentrated and washed encapsulated treatment component transferred to another vessel for further suspension in the carrier.

Administration

As described above, treatment compositions including one or more components that are encapsulated, or a combination of encapsulated and unencapsulated materials, may be administered as a beverage. The beverage may include water, the active components described above in solution or suspension, and other components such as flavorings or electrolytes. The materials other than water may be stored in a dry form to be added to a quantity of water shortly before use. The invention includes a method of providing a dry combination of encapsulated materials as described above, adding the dried combination to a container of water to form a suspension, and drinking the suspension.

I refer in this specification to embodiments as “one embodiment,” “an embodiment,” “another embodiment,” etc. These references indicate that the embodiments described can include a particular feature, structure, or characteristic, but every embodiment does not necessarily include every described feature, structure, or characteristic. Where I describe a particular feature, structure, or characteristic is described in connection with an embodiment, it should be understood that it is within the knowledge of one skilled in the art to include such feature, structure, or characteristic in connection with other embodiments whether or not explicitly described. Any of the features, functions, materials, or construction details may be combined with any other of the described features, functions, materials, or construction details. The scope of the invention is not limited to those described embodiments but includes such equivalents as would be apparent to a person skilled in the art upon reading this disclosure. Further, where specific examples are given, the skilled practitioner may understand the particular examples as providing particular benefits such that the invention as illustratively disclosed herein suitably may be practiced in the absence of any element which is not specifically disclosed herein or within that particular example. While the foregoing is directed to certain preferred embodiments of the present invention, other and further embodiments of the invention may be devised without departing from the basic scope of the invention. I intend the scope of any appended claims to encompass such alternative embodiments.

I claim:

1. A composition comprising nicotinamide mononucleotide (NMN), pterostilbene, and xanthohumol.

2. The composition of claim 1, including about 25-500 mg NMN, about 10-100 mg pterostilbene, and about 4-100 mg xanthohumol.

3. The composition of claim 2, wherein the NMN, the pterostilbene, and the xanthohumol are suspended or dissolved in 200-1000 mL water.

4. The composition of claim 1, wherein one or more of the NMN, the pterostilbene, and the xanthohumol are encapsulated.

5. The composition of claim 4, wherein one or more of the NMN, the pterostilbene, and the xanthohumol are dried.

6. The composition of claim 5, wherein the NMN, the pterostilbene, and the xanthohumol are suspended or dissolved in water.

7. A composition comprising an aqueous suspension of an encapsulated active component, wherein the active component includes one or more of NMN, pterostilbene, and xanthohumol.

8. The composition of claim 7, wherein the encapsulated active component has a capsule median diameter in the range of about 20 nm to about 500 nm.

9. The composition of claim 7, wherein the active component includes two or more of NMN, pterostilbene, and xanthohumol.

10. The composition of claim 7, wherein the active component includes NMN, pterostilbene, and xanthohumol.

11. The composition of claim 10, wherein at least two of the NMN, the pterostilbene, and xanthohumol are separately encapsulated.

12. The composition of claim 7, wherein the encapsulated active component comprises a capsule including one or more of a liposome or a polymer capsule.

13. The composition of claim 12, wherein the polymer capsule includes one or more of an alginate, a polysaccharide, a protein, a synthetic polymer, or a derivative of any of these.

14. The composition of claim 12, wherein the capsule includes a targeting material.

15. A method of administering a treatment composition comprising the steps of providing a first composition including a dry encapsulated active component, wherein the active component includes one or more of NMN, pterostilbene, and xanthohumol;
suspending a predetermined amount of the first composition in a predetermined volume of water to form a beverage; and
drinking the beverage.

16. The method of claim 15, wherein the predetermined volume is about 200 to about 1000 mL.

17. The method of claim 15, wherein the first composition includes an unencapsulated active component.

18. The method of claim 17, wherein the unencapsulated active component includes a flavoring or an electrolyte.
